(19) United States
(12) Patent Application Publication
(10) Pub. No.: US 2017/0290651 A1
(43) Pub. Date: Oct. 12, 2017

(54) Surface Structuring Method for an Implant, A Counter-Body, and an Implant

(71) Applicant: Technische Universität Darmstadt, Darmstadt (DE)

(72) Inventors: Markus König, Monsheim (DE); Anne Martin, Darmstadt (DE); Herbert Scheerer, Heppenheim (DE); Emmerich Eckert, Griesheim (DE); Matthias Oechsner, Darmstadt (DE)

(21) Appl. No.: 15/481,745

(22) Filed: Apr. 7, 2017

(30) Foreign Application Priority Data
Apr. 8, 2016 (DE) 10 2016 106 492.0

(51) Int. Cl. A61F 2/00; C23C 14/02; B29C 59/02; C23C 14/20; A61F 2/02; A61F 2/30

(52) U.S. Cl. CPC A61F 2/0077; A61F 2/02; A61F 2/30767; A61F 2/3094; B29C 59/022; C23C 14/20; C23C 14/02; A61F 2002/3093; A61F 2002/30971; A61F 2002/3098; A61F 2002/30981; A61F 2240/001; B29L 2031/7532

(57) ABSTRACT

A method for structuring a surface of an implant (100) made from a plastic material by means of a counter-body (200) comprises the following steps: providing (S110) of a counter-body (200) including a surface (210); forming (S120) of a first surface structure (212) on the surface (210) of the counter-body, wherein a first surface structure (212) comprises a non-regular, randomly distributed pattern; and forming (S130) of a second surface structure (112) on the implant (100) by using the counter-body (200), wherein the second surface structure and the first surface structure (212) are complementary to each other.

The present invention relates to a method for structuring a surface of an implant, a counter-body, an implant and, in particular, to a polymer implant with a coated surface structure.

### BACKGROUND

For many implants, such as spine disc implants, it is important and desired to ensure a fast healing process without any complications. For example, a purposeful creation of microstructures free of foreign substances on a plastic implant surface aims at an integration of desired cell types (e.g. osteoblasts). Hereby, a fast, mechanically reliable connection of the implant is achieved and the patient may be re-mobilized faster. Thus, in addition the risk of secondary diseases or complications (for example inflammation, circulation problems etc.) is minimized.

For metal-based implants a variety of solutions to this problem are already available, wherein the respective surfaces are for example processed by a sandblasting treatment in a way that the desired effect is achieved. However, there are no comparable products for plastic-based implants.

Here is a need for a surface structuring method of implants including a plastic material.

### SUMMARY

The technical object described above is achieved by a surface structuring method according to claim 1, a counter-body according to claim 12, and an implant according to claim 13. The dependent claims relate to advantageous developments of the method according to claim 1.

The present invention relates to a surface structuring method of an implant made from plastic material by means of (at least) one counter-body. The method includes the steps: providing of a counter-body including a surface, forming of a first surface structure on the surface of the counter-body, wherein the first surface structure comprises a non-regular, randomly distributed pattern, and forming of a second surface structure on the implant by using the counter-body, wherein the second surface structure and the first surface structure may be complementary to each other. It is also possible to use a three-tier method, for example. In this case however, the created structure would not be complementary to the original, but still complementary to the counter-body which is produced in the intermediate step from the original. Thus, the counter-body is to be the body, which forms the implant surface by direct contact.

An implant may denote any body which may be integrated in an organism and which assumes the function of natural human body materials (for example a spinal disc). Here, the implant does not need to be made completely from plastics, but may also comprise several material classes, but the implant surface to be structured has to be fabricated from a plastic material. The surface of the counter-body comprises a minimum hardness, which is purposefully chosen such that the surface of the implant may be structured by using the surface of the counter-body, whereto further exemplary embodiments define a variety of possibilities.

The non-regular, randomly distributed pattern is in particular a non-repeating pattern, and will thus not be applied by a pressure roller etc. on the implant. The random distribution of the pattern relates to the size of individual pattern elements, for example, and also to the shape of the pattern elements or the orientation of the pattern elements, which may be arbitrary or random.

The technical object described above is achieved by a two-tier method for surface structuring of implants made of plastic material. Here, the first step comprises a structuring of a counter-body, and the second step comprises an imprint or forming of the structure of the counter-body on the implant surface. This may be performed by a reshaping process, an injection molding or extrusion process, for example. By applying the intended surface structure on a hard counter-body, which is easy to clean, an incorporation of foreign particles in the plastic surface is prevented. In addition, a counter-body allows the material for the implant to be selected according to biological factors (for example organic compatibility for fast healing).

The plastic material may for example be based on polymer, and comprise the following materials: the material of the PEK group, PEK or PMMA. In particular, the implant material may also be a non-metal, and comprises no ceramic or other material having a high hardness. For example, the implant material may also be a gel or hydroxylapatite. In further exemplary embodiments the forming of the second surface structure comprises a heating of the implant. The heating of the implant particularly enables the second implant surface to be softened accordingly, thus a structuring in an average structural size between 10 μm and 50 μm is enabled or is facilitated.

In further exemplary embodiments the surface of the counter-body comprises a hard body as compared to the implant material. Suitable materials for the counter-body may be, for example, ceramic, metal, a hard-coated base body, copper, steel, titanium or another metal compound having a minimum hardness in order to accordingly structure the plastic material of the implant on the surface thereof by using said counter-body.

In further exemplary embodiments the step of forming a first surface structure comprises a separation process (for example blasting, abrasive, milling, lathing, eroding) or a forming process (for example rolling or forging). However, the separation process for forming a surface structure of the counter-body may also comprise a sand-blasting process, wherein particles in different sizes may be used to create a randomly distributed pattern. Here, a random pattern is created in a natural way. The method may further comprise a purification step, which cleans the first surface structure, before the second surface structure is formed.

In further exemplary embodiments the step of forming a second surface structure comprises an imprint or reshaping process. The reshaping process may comprise an overmolding process or a plastic reshaping or any other formation process, which uses a counter-body for formation.

In further exemplary embodiments, forming of the first surface structure comprises a forming of structural elements in different sizes, wherein a maximum medium dimension of the structural elements varies between 0.8 μm and 500 μm, wherein at least 5% comprise at least 1 μm. An average structural size (of all structural elements or at least 5% thereof) may comprise 50 μm or less (for example less than 25 μm) and at least 10% may comprise about 1 μm. An average structural size (average of all structural element sizes) may for example lie between 5 μm and 100 μm (or between 10 μm and 25 μm). An (average) dimension of a structural element may, for example, be mathematically defined as root of the surface area (because every structural element comprises a surface-like dimension and thus a surface area on the surface thereof).

In further exemplary embodiments the forming of the first surface structure on a surface of the counter-body comprises a subsequent forming of a protection layer against wear in order to protect the formed first surface structure. The protection layer against wear may for example comprise a hard material layer configured to adequately protect the counter-body against wear.

In the further exemplary embodiments the material of the counter-body has a minimum hardness which is at least about 5 times or 10 times higher than the hardness of the implant on the surface that is to be structured.

In further exemplary embodiments the method comprises an application of an additional substance between the step of forming the first surface structure and the forming of the second surface structure, wherein the additional substance is adapted to facilitate the separation of the counter-body from the implant after forming of the second surface structure. The additional substance may for example comprise a film, a powder, or a varnish, and is added between the counter-body and the surface of the implant, which is to be structured, to enhance the removal of the counter-body from the implant surface. In addition, a mechanical or functional property of a peripheral layer region is achieved by the additional substance, for example. Finally, a conditioning of the implant surface for a subsequent treatment (such as coating) may be performed due to the additional substance.

In further exemplary embodiments the step of forming a second surface structure comprises forming of a coating on the plastic material of the implant, wherein the coating may in particular comprise a metal (for example titanium, tantalum) and may be formed by a physical vapor deposition (PVD) or by sputtering.

Optionally, the step of forming a coating may also comprise an activation of the plastic surface by a physical or chemical treatment of the surface. The surface activation refers to the breaking up of physical structures or the creation of geometrical structures.

Optionally, the step of forming the second surface structure may also comprise the activation of the second surface structure (for example, after developing the surface layer). A surface activation may also denote a breaking up of an atomic surface structure in order to facilitate integration with the adjacent organic material. This may be performed by an acid treatment, for example.

The coating and activation steps may also be combined. For example a sputtered titanium may be treated with acid in order to form an activated surface coating, which may enhance the healing process in the body, in a single step.

The present invention also relates to a counter-body to form a structural surface on a plastic implant. The counter-body comprises a surface structure with a non-regular, randomly distributed pattern, which is complementary to the structured surface of the implant and has a higher hardness than the plastic implant in the area of the structured surface.

The present invention relates also to an implant comprising a plastic material wherein the plastic material comprises a surface structure with a non-regular, randomly distributed pattern of structural elements with a maximum dimension of structural elements ranging between 0.5 μm and 300 μm, and an average structural size between 0.1 μm and 100 μm in the plastic material.

Exemplary embodiments advantageously enable a simple and fast structuring of plastic implant surfaces in high quantities. In particular, exemplary embodiments are suitable for injection molding, reshaping in a way that prevents an inclusion of impurities in the implant. In addition, exemplary embodiments are suitable for direct implantation or for a further enhancement by using a deposition method, wherein for example a PVD deposition method may be used.

### BRIEF DESCRIPTION OF FIGURES

The exemplary embodiments of the present invention will become clearer by means of the following detailed description and the appended figures of the different exemplary embodiments, which however should not be interpreted as limiting the disclosure to the specific embodiments, but are only intended as an explanation and to enhance understanding.

FIG. 1 shows a flow diagram of a method for structuring the surface of an implant.

FIG. 2 shows further optional process steps for creating a surface structure.

FIG. 3 shows an exemplary embodiment for creating a counter-body with a desired surface structure.

FIG. 4 shows an exemplary surface of a counter-body and a structured surface of the implant body that is formed complementarily thereto.

### DETAILED DESCRIPTION

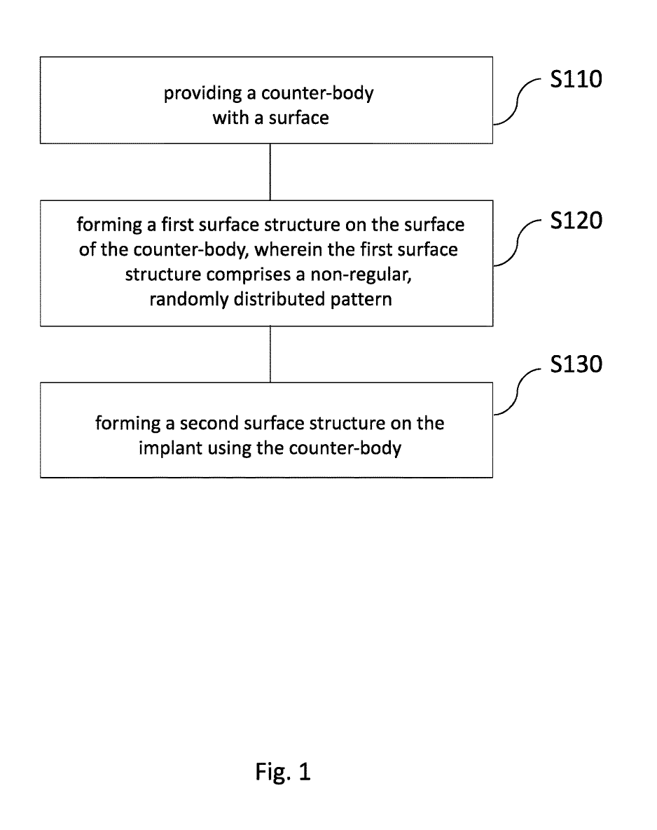

FIG. 1 shows a flow diagram of a method for structuring the surface of an plastic implant by using a counter-body. The method comprises providing S110 a counter-body with a surface and a forming S120 of a first surface structure on the surface of the counter-body, wherein the first surface structure comprises a non-regular, randomly distributed pattern. The method further comprises a forming S130 of a second surface structure on the implant by using the counter-body, wherein the second surface structure and the first surface structure are complementary to each other.

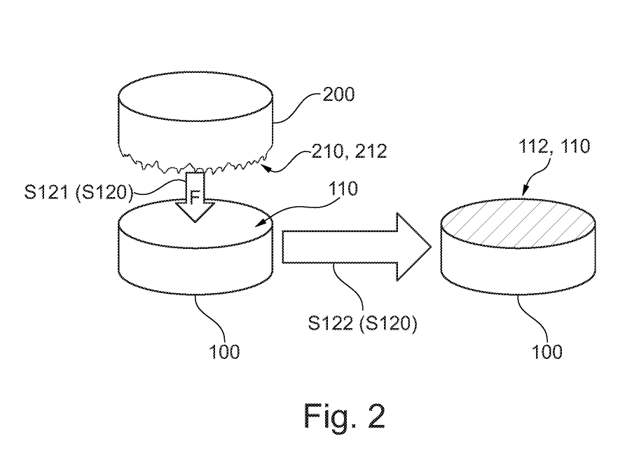

FIG. 2 shows further optional process steps to create a surface structure on the plastic surface 110 of the implant 100. The counter-body 200 with the intended surface structure 212 on a surface 210 is thus pressed on a surface 110 of the implant body 100, which is to be structured, using a force F. In the exemplary embodiment shown, the step S120 thus comprises a pressing-on operation S121. Then a reshaping process or imprint process is performed, which results in applying the surface structure 212 of the counter-body 200 on the surface 110 of the implant 100, for example. As a result, the structured implant surface 112 is formed on the implant 100.

As part of step S120, the reshaping process may comprise a heating S122 of the implant 100 or at least of the implant surface 110 the counter-body surface 210, for example. Optionally, a corresponding pressing force F may also be adjusted; thus the structured surface 212 of the counter-body 200 may be pressed into the plastic implant 100. As a result, the surface structure 110 of the implant 100 is complementary to the surface structure 212 of the counter-body 200.

Optionally, a transfer of the surface structure 212 to an implant surface 110 may be performed by a reshaping process at room temperature, wherein a sufficiently high pressure or force F is to be applied, for example, in order to form a structured surface 112 on the plastic surface no. During execution of the reshaping process S121, S122 at an increased temperature, the temperature may be selected such that the plastic material of the implant 100 is correspondingly weakened in order to facilitate the forming of the surface structure 112. Optionally, it is further possible to use the structured counter-body 200 as a form, or as a stamp, or imprint body, thus the surface structure 112 will be created or transferred on the implant surface 110 by using a press and by plastic reshaping.

In this reshaping process S121, S122, a minimum contact time between the counter-body 200 and an implant 100 may be observed, which for example may be 3 s, 10 s, or 10 min, or any duration may be chosen, until the desired result is achieved.

For example, the counter-body 200 may comprise a metallic material (for example copper or steel), so at least the surface 210 with the structuring 212 has a sufficient hardness to reshape the surface 110 of the implant part by the counter-body 200, without reshaping the counter-body 200 by itself (for example when applying the force F).

An additional substance is inserted between the counter-body 200 and the surface 110 to be structured, where appropriate. The additional substance may for example comprise a film, a powder, or a varnish, and is intended to enhance removability. Thus, after forming the structured surface 112, a removal of the counter-body 200 from the implant body 100 is facilitated due to the additional substance. In addition, such an additional substance may help to achieve a change of the mechanical or functional properties of the peripheral layer region (that is, the top implant area).

It is further possible to condition the surface 110 of at least one implant section for a subsequent treatment, which may comprise a coating, for example. A coating may be performed by using a PVD method, for example, wherein a metallic material (for example titanium) may be formed on the plastic material. The structured surface 112 or the coating may be finally or concurrently activated to enhance an organic adaption process to adjacent organic material. The surface activation may be performed by an acid treatment or physical etching.

Exemplary embodiments thus allow an adapted microstructure for implant bodies 100 to be created from plastic material, wherein the microstructure may be adapted to the deposition procedure and to biologically relevant aspects. Exemplary embodiments enable for example implants without particles which allow a cell differentiation, an adhesion, and increased vitality of certain cell types, such as osteoblasts.

In further exemplary embodiments the structured surface 212, 210 of the counter-body is used as a tool for an injection molding procedure or an extrusion procedure, wherein the structured surface 212, 210 is transferred to an implant section 110 by a reshaping process.

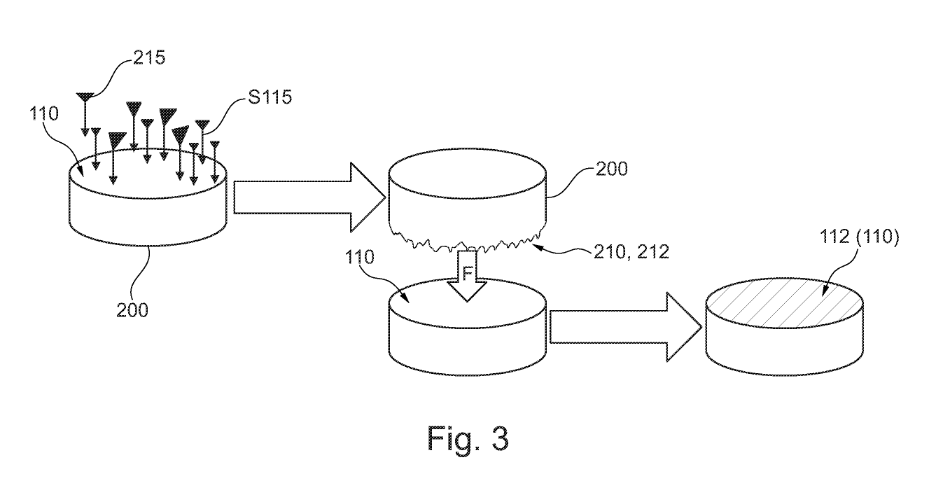

FIG. 3 shows a further exemplary embodiment, wherein the reshaping process of the implant surface 110 be executed as described in FIG. 2. In the exemplary embodiment of FIG. 3 the surface 210 of the counter-body 200 is initially structured by an exemplary machining process S115. In the process S115 particles 215 may be applied on the surface 210 of the counter-body 200 with a sufficiently high energy, wherein the energy of the particle 215 is chosen at such a level that an intended structuring of the surface 210 is performed (that is, the intended surface structure 212 is created). By means of the structured surface 212 thus created on the counter-body 200, the implant surface 110 may be structured correspondingly in the reshaping process in a complementary way, as described in FIG. 2.

Particles 215 of the sandblasting procedure may have a random size, so the energies with which particles impinge on the surface 210 are also distributed randomly and thus a random, non-repeating surface structure is formed on the surface 210 of the counter-body 200. This random structural distribution on the surface 210 has advantages in the biological integration process in organisms. As a further method of structuring of the counter-body separation processes may be used, which for example include milling, lathing, eroding, or also reshaping processes, like for example blasting, rolling, or forging.

Here, a typical length scale of the created microstructures lies in the range of submicrometers to some hundred μm (for example 0.3 μm . . . 300 μm) or between 0.5 and 240 μm. This length scale may be deliberately chosen according to a determination of the particle size in the exemplary sandblasting treatment.

It is advantageous for the surface 210 of the counter-body 200 to be comparatively hard (for example a metallic surface, for example made from copper or steel), so the transfer of the structured surface 212 to a plastic surface 110 may be performed without reshaping the structured counter-body surface 212.

The counter-body surface 212 is protected against wear after structuring by a wear protection layer, if appropriate. The protection layer against wear may for example be a hard material layer, which causes a further hardening of the structured surface 212.

The counter-body 200 has an up to 10 times higher hardness than the material of the implant 100. In particular, if the surface 210 of the counter-body 200 has been structured by sandblasting, due to the significantly higher hardness of the counter-body 200 no particles 215 are included in the counter-body 200 or only to a minor degree. In addition, potential, still adhering particles may be removed from the structured surface 212 in a cleaning process, before the structured surface 212 is transferred from the counter-body 200 to a surface 110 of the implant body 100. Hereby, also a higher hardness or strength of the material of the counter-body 200 compared to the plastic material of the implant 100 is advantageous, as a simple mechanical cleaning without changing the surface structure 212 becomes possible.

It is important, both for the manufacturing of the (optional) coating of the implant 100 and also for organic compatibility to create the microstructure without incorporation or adhesion of any foreign substances on the implant surface 110. This issue is prevented in exemplary embodiments as not the implant 100, but the counter-body 200 is structured by means of an exemplary sandblasting treatment. The particles (impurities) thus only contaminate the counter-body 200—but not the implant 100—and may easily be removed from the counter-body 200.

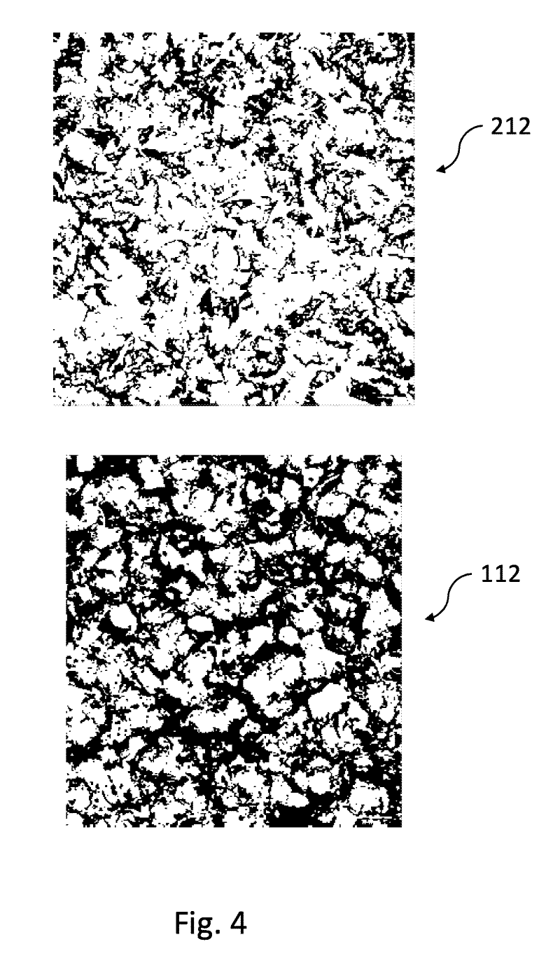

FIG. 4 shows an exemplary structured surface of the counter-body 212 (at the top of FIG. 4) and the structured surface 112 of the implant body 100 formed hereto in a complementary way (at the bottom of FIG. 4). On the structured surface shown, light portions are depressions, for example, and dark portions elevations, or vice versa, wherein elevations on the counter-body 200 result in depressions on the implant surface 110.

Compared to conventional processes the exemplary embodiments of the present invention provide the following benefits.

When using conventional processes to structure metallic implant surfaces 110 no foreign substances are incorporated in the implant 100. However, studies have shown that the lower hardness of plastic materials may cause an embedding of blasted particles on a substrate surface. For example, the material PEEK has a hardness degree of 15 to 38 HV as compared to titanium with 300 to 400 HV. Such particles have to be considered as very critical under biological aspects, as a detachment of particles from the surface may lead to an inflammatory reaction and also to necrosis and to granular tissue. In addition, in conventional processes the incorporated particles have to be considered as detrimental for a subsequent coating of the surfaces. They cause for example a faulty growth of layers and a detachment of the layer during removal. In addition, they prevent a physical activation of the surface, thus impacting the adhesion of the layer.

Exemplary embodiments of the present invention handle this problem by a two step approach, which prevents the incorporation of particles in the implant body 100.

The features of the invention disclosed in the specification, the claims and the figures may be significant for the realization of the invention be it as single features or also in any combination thereof.

### LIST OF REFERENCE NUMBERS

100 Implant
110 Implant surface
112 Second implant surface
200 Counter-body
210 Surface of the counter-body
212 First surface structure
215 Particle

1. A method for structuring a surface of an implant (100) made from plastics by means of a counter-body (200), comprising the following steps:
providing (S110 ) of a counter-body (200) comprising a surface (210);
forming (S120) of a first surface structure (212) on the surface (210) of the counter-body, wherein the first surface structure (212) comprises a non-regular, randomly distributed pattern; and
forming (130) of a second surface structure (112) on the implant (100) using the counter-body (200), wherein the second surface structure and the first surface structure (212) are complementary to each other.

2. The method according to claim 1, wherein the forming (S130) of the second surface structure (112) comprises a heating (S122) of the implant (100).

3. The method according to claim 1, wherein the surface (210) of the counter-body (200) comprises a metal.

4. The method according to claim 1, wherein the step of forming (S120) the first surface structure (212) comprises a separation process or a reshaping process.

5. The method according to claim 1, wherein the step of forming (S130) the second surface structure (112) comprises an imprint process (S121) or a reshaping process (S122).

6. The method according to claim 1, wherein the forming (S120) of the first surface structure comprises a forming of structural elements in different sizes, wherein a maximum average dimension of the structural elements varies between 0.8 μm and 500 μm, wherein at least 5% comprise at least 1 μm, and a middle structural size amounts to 50 μm or less, and wherein at least 10% comprise about 1 μm.

7. The method according to claim 1, wherein the forming (S120) of the first surface structure (212) on the surface (210) of the counter-body (200) comprises a subsequent forming of a protection layer against wear in order to protect the first surface structure (212).

8. The method according to claim 1, wherein the counter-body (200) comprises a minimum hardness which is at least 5 times or 10 times higher than the hardness of the implant (100) on the surface (110).

9. The method according to claim 1, comprising an application of an additional substance between the step of forming (S120) of the first surface structure (212) and the forming (S130) of the second surface structure (112), wherein the additional substance is adapted to facilitate a separation of the counter-body (200) from the implant (100) after forming (S130) the second surface structure (112).

10. The method according to claim 1, wherein the step of forming (S130) the second surface structure (112) comprises a development of a coating on the implant (100), wherein the coating comprises in particular a metal or any other class of material.

11. The method according to claim 1, wherein the step of forming (S113) the second surface structure (112) comprises an activation of the second surface structure (112).

12. A counter-body (200) for forming a structured surface (112) on a plastic implant (100), including:
a surface structure (212) including a non-regular, randomly distributed pattern, which is complementary to a structured surface (112) of the implant (100), which has to be formed, and comprises a higher hardness than the plastics implant (100) in the region of the surface (110) to be structured.

13. An implant (100) comprising plastic material, wherein in the plastic material a surface structure (112) with the non-regular, randomly distributed pattern of structural elements is formed, which comprise maximal dimensions in a range of 0.5 μm to 300 μm, and an average structural size between 5 μm and 20 μm.
